(19) United States
(12) Patent Application Publication
(10) Pub. No.: US 2023/0210960 A1
(43) Pub. Date: Jul. 6, 2023

(54) NEW USE OF CMTR1 HAVING SIRNA PRODUCTION AND FUNCTION ENHANCING ACTIVITY

(71) Applicant: Korea University Research and Business Foundation, Seoul (KR)

(72) Inventors: Young Sik LEE, Seoul (KR); Seung Jae LEE, Seoul (KR); Jae Sang HONG, Seoul (KR); Do Hwan LIM, Seoul (KR)

(73) Assignee: Korea University Research and Business Foundation, Seoul (KR)

(21) Appl. No.: 18/000,996

(22) PCT Filed: Jun. 3, 2021
(86) PCT No.: PCT/KR2021/006979

§ 371 Date: Dec. 7, 2022

(30) Foreign Application Priority Data
Jun. 18, 2020 (KR) 10-2020-0074360
Sep. 2, 2020 (KR) 10-2020-0111757

(51) Int. Cl. A61K 38/45; C12N 15/113; C12N 9/10; C12N 15/63

(52) U.S. Cl. CPC A61K 38/45; C12N 15/113; C12N 9/1007; C12Y 201/01057; C12N 15/63

(57) ABSTRACT

The present disclosure relates to a novel use of CMTR1 having the activity of enhancing the production and function of siRNA, and more particularly, to a composition for enhancing the production of siRNA including a cap1 2′-O-ribose methyltransferase (CMTR1) protein as an active ingredient, and a composition for enhancing the gene silencing activity by siRNA, and also to a method for producing siRNA for gene silencing in vitro. According to the present disclosure, the CMTR1 may enhance the production of siRNA for gene silencing and at the same time, ultimately enhance the production and function of siRNA without artificial chemical modification of siRNA by enhancing the formation of holo-RNA-induced silencing complex (RISC) that acts on silencing of a target gene in an RNAi mechanism. Therefore, the CMTR1 of the present disclosure can be usefully used in the development of pharmaceuticals using siRNA as a therapeutic agent.

### TECHNICAL FIELD

The present disclosure relates to a novel use of cap1 2′-O-ribose methyltransferase (CMTR1) with the activity of enhancing production and function of siRNA.

### BACKGROUND ART

RNA interference (RNAi) is an evolutionarily conserved phenomenon in eukaryotes, and has a function of regulating gene expression by degrading sequence-complementary mRNA or inhibiting the translation into protein by RNA (siRNA or miRNA) having a length of 21 to 23 nucleotides. RNAi is closely related to various biological processes, such as development and physiology of living organisms, differentiation, proliferation, and death of cells, genome stability through activity inhibition of transposon, and the like. In addition, as RNAi is closely associated with resistance to viruses and various human diseases, studies for application to drug development, disease diagnosis/treatment, and agriculture and livestock fields have been actively conducted.

As a representative RNAi mechanism so far, double-stranded RNA (dsRNA) or hairpin miRNA precursor (pre-miRNA) expressed from the genome of a cell is recognized and processed by Dicer in the cytoplasm to be converted into two classes of small RNAs, siRNA and miRNA, and then binds to an RNA-induced silencing complex (RISC), which has an Argonaute (Ago) family protein as a key component to induce cleavage/degradation of sequence-complementary mRNA or to inhibit its translation to a protein, thereby suppressing the expression of the corresponding gene.

In addition, it has been reported that various protein factors are involved in such an RNAi phenomenon, and for example, the Dicer is known to play an important role in the formation of RISC as well as the production of siRNA and miRNA. The Dicer is known to interact with various factors (TRBP, PACT, R2D2, or Logs) with dsRNA-binding domains as needed and to interact with Ago family proteins. Another major RNAi factor, Ago, additionally interacts with various proteins, such as FMRP, Gemini, Gemin4, MOV10, and TNRC6B, and is known to regulate the RNAi phenomenon.

Recently, research to use siRNA as a disease therapeutic agent has been actively conducted, and in 2018, FDA approved Onpattro, patisiran as an RNAi therapeutic agent of Alnylam Pharmaceuticals as a therapeutic agent of polyneuropathy in adult patients with hereditary transthyretin-mediated (hATTR) amyloidosis. Onpattro is an siRNA therapeutic agent and is the first and unique therapeutic agent for polyneuropathy approved by the FDA. In addition, many disease therapeutic agents using gene suppression technology through siRNA are in the clinical stage.

siRNA expressed in somatic cells of various mammals as well as Drosophila is generated from cis-dsRNA, trans-dsRNA, and precursors with long hairpin structures, and has been reported to maintain the stability of the genome by inhibiting the activity of the transposon, and to perform an important biological function of regulating the expression of protein-coding genes.

In addition to major RNAi regulators such as Dicer and Ago proteins, various additional RNAi regulators are likely to perform a function for regulating gene expression in a tissue- or cell-specific manner. Accordingly, the identification of the biological functions of the corresponding RNAi regulators may play an important role in improving the production and ability of disease therapeutic agents through siRNA in the future.

On the other hand, siRNA plays an important role in the RNAi phenomenon, but has various problems to be used as a therapeutic agent. First, siRNA is easily degraded by enzymes and rapidly removed from the kidney due to its small size, and is recognized through Reticuloendothelial system (RES)/mononuclear phagocytic system (MPS) by an opsonic action in the liver and pancreas during blood circulation to be easily removed, so that there is a problem that the therapeutic affect is reduced. In addition, there is a problem in that siRNA is not easy to move between cell membranes due to a negative charge (40 to 50 negative phosphate charge).

Accordingly, technologies for efficient delivery and stability enhancement of siRNA have been developed by improving these problems, including a method for enhancing stability by substituting 2′-OH of siRNA sugar with a chemical group such as 2′-O-methyl, 2-H, and 2-fluoro, or for increasing in vivo permeability of siRNA by using cationic polymers such as liposome, polyethylenimine (PEI), poly-L-lysine (PPL), chitosan, and dendrimers.

However, a conventionally developed technology is to improve the function of siRNA through chemical modification, and thus, there is a need for development of a new technology capable of discovering a regulator capable of controlling the function of siRNA in a cell without chemical modification and improving the production and function of siRNA through the corresponding regulator.

### DISCLOSURE OF INVENTION

### Technical Problem

Accordingly, the present inventors first identified that cap1 2′-O-ribose methyltransferase (CMTR1) had the activity of enhancing the production and function of siRNA.

An object of the present disclosure is to provide a composition for enhancing the production of siRNA including a cap1 2′-O-ribose methyltransferase (CMTR1) protein as an active ingredient.

Another object of the present disclosure is to provide a composition for enhancing the gene silencing activity by siRNA including a cap1 2′-O-ribose methyltransferase (CMTR1) protein as an active ingredient.

Yet another object of the present disclosure is to provide a method for producing siRNA for gene silencing in vitro, including treating a cell with an expression vector containing a gene encoding an amino acid sequence of CMTR1.

### Solution to Problem

Therefore, the present disclosure provides a composition for enhancing the production of siRNA including a cap1 2′-O-ribose methyltransferase (CMTR1) protein as an active ingredient.

In an example embodiment of the present disclosure, the CMTR1 protein may consist of an amino acid sequence of SEQ ID NO: 1 or SEQ ID NO: 5.

In an example embodiment of the present disclosure, the CMTR1 protein may be encoded by a nucleotide sequence of SEQ ID NO: 2 or SEQ ID NO: 6.

In an example embodiment of the present disclosure, on an RNAi pathway triggered by dsRNA, the CMTR1 may increase the production of siRNA from dsRNA through formation of a cap1 structure by 2′-O-ribose methylation and increase the gene silencing activity by promoting the formation of a holo-RNA-induced silencing complex (RISC) capable of cleaving a target mRNA.

In an example embodiment of the present disclosure, a carboxyl-terminal region of the CMTR1 may bind to a carboxyl-terminal region of R2D2 in the holo-RISC complex.

In an example embodiment of the present disclosure, the carboxyl-terminal region of the CMTR1 may consist of the 388th to 788th amino acid sequence of SEQ ID NO: 1, and the carboxyl-terminal region of R2D2 may consist of the 237th to 311th amino acid sequence in a R2D2 amino acid sequence of SEQ ID NO: 3.

In an example embodiment of the present disclosure, in the cap1 2′-O-ribose methyltransferase (CMTR1), a gene encoding the CMTR1 amino acid sequence of SEQ ID NO: 1 or SEQ ID NO: 5 may be inserted into an expression vector.

Further, the present disclosure provides a composition for enhancing the gene silencing activity by siRNA including a cap1 2′-O-ribose methyltransferase (CMTR1) protein as an active ingredient.

In an example embodiment of the present disclosure, the CMTR1 protein may consist of an amino acid sequence of SEQ ID NO: 1 or SEQ ID NO: 5.

In an example embodiment of the present disclosure, the CMTR1 protein may be encoded by a nucleotide sequence of SEQ ID NO: 2 or SEQ ID NO: 6.

In an example embodiment of the present disclosure, on an RNAi pathway triggered by dsRNA, the CMTR1 may increase the production of siRNA from dsRNA through formation of a cap1 structure by 2′-O-ribose methylation to enhance the gene silencing activity, and the carboxyl-terminal region of CMTR1 may bind to the carboxyl-terminal region of R2D2 in the holo-RISC complex to promote the formation of a holo-RNA-induced silencing complex (RISC) capable of cleaving a target mRNA for gene silencing.

In an example embodiment of the present disclosure, the carboxyl-terminal region of the CMTR1 may consist of the 388th to 788th amino acid sequence of SEQ ID NO: 1, and the carboxyl-terminal region of R2D2 may consist of the 237th to 311th amino acid sequence in an R2D2 amino acid sequence of SEQ ID NO: 3.

Further, the present disclosure provides a method for producing siRNA for gene silencing in vitro, including treating a cell with an expression vector containing a gene encoding a CMTR1 amino acid sequence of SEQ ID NO: 1 or SEQ ID NO: 5.

In an example embodiment of the present disclosure, the gene may consist of a nucleotide sequence of SEQ ID NO: 2 or SEQ ID NO: 6.

### Advantageous Effects of Invention

The present disclosure relates to a composition for enhancing the production of siRNA including a cap1 2′-O-ribose methyltransferase (CMTR1) protein as an active ingredient, and a composition for enhancing the gene silencing activity by siRNA, and also relates to a method for producing siRNA for gene silencing in vitro. According to the present disclosure, the CMTR1 may enhance the production of siRNA for gene silencing and at the same time, ultimately enhance the production and function of siRNA without artificial chemical modification of siRNA by enhancing the formation of a holo-RNA-induced silencing complex (RISC) that acts on silencing of a target gene in an RNAi mechanism. Therefore, the CMTR1 of the present disclosure can be usefully used in the development of pharmaceuticals using siRNA as a therapeutic agent.

### BRIEF DESCRIPTION OF DRAWINGS

The foregoing and other objects, features, and advantages of the disclosure, as well as the following detailed description of the example embodiments, will be better understood when read in conjunction with the accompanying drawings. For the purpose of illustrating the disclosure, there is shown in the drawings an exemplary embodiment that is presently preferred, it being understood, however, that the disclosure is not intended to be limited to the details shown because various modifications and structural changes may be made therein without departing from the spirit of the disclosure and within the scope and range of equivalents of the claims. The use of the same reference numerals or symbols in different drawings indicates similar or identical items.

FIG. 1 illustrates results of confirming eye colors of a dCMTR1^{W231X} mutant having a GMR-wIR transgene as a genetic background and having a mutation on a Drosophila X chromosome by EMS treatment, an Ago2⁴¹⁴ mutant and wild-type Drosophila.

FIG. 2 illustrates results of identification of Drosophila CMTR1 (dCMTR1) identified as a gene related to gene silencing in the present disclosure and orthologs having the same function as dCMTR1 in respective eukaryotes.

FIG. 3 illustrates results of confirming GMR-wIR-induced white RNAi according to the presence or absence of a cap1 2′-O-ribose methyltransferase (2′-O-MTase) activity. FIG. 3A illustrates eye colors of Drosophila as results for (A) dCMTR1+; GMR-GAL4, GMR-wIR/+(B) dCMTR1^{W231X}; GMR-GAL4, GMR-wIR/+(C) dCMTR1^{W231X}; GMR-GAL4, GMR-wIR/UAS-dCMTR1 (D) dCMTR1^{W231X}; GMR-GAL4, GMR-wIR/UAS-dCMTR1^{K179A} (E) dCMTR1^{W231X}; GMR-GAL4, GMR-wIR/UAS-hCMTR1 (F) dCMTR1^{W231X}; GMR-GAL4, GMR-wIR/UAS-dCMTR1^{K239A}, and FIG. 3B illustrates an absorbance measurement result thereof in a graph.

FIG. 4 illustrates results of confirming 2′-O-ribose methylation in a dCMTR1^{W231X} mutant through 2D-TLC, in which FIG. 4A illustrates a 2D-TLC result of standard mononucleotides according to the presence or absence of 2′-O-ribose methylation and FIG. 4B illustrates a 2D-TLC result of a wild-type and a dCMTR1^{W231X} mutant.

FIG. 5 illustrates results of confirming 2′-O-ribose methylation through overexpression of catalytic dead proteins of human CMTR1 (hCMTR1) and dCMTR1 in the dCMTR1^{W231X} mutant through 2D-TLC.

FIG. 6 illustrates results of confirming an increase in gene silencing according to a cap1 structure of dsRNA, in which FIG. 6A illustrates no cap structure (no cap), and structures of cap0 and cap1, and FIG. 6B illustrates a result of analyzing the silencing activity of a GFP gene thereof.

FIG. 7 illustrates results of confirming association of dCMTR1 with siRNA production through northern blotting.

FIG. 8 illustrates results of confirming the cleavage reducing activity of siRNA-induced target mRNA in a dCMTR1^{W231X} mutant.

FIG. 9 illustrates results of confirming a reduction in RISC complex formation in the dCMTR1^{W231X} mutant through native gel electrophoresis.

FIG. 10 illustrates results of confirming a reduction in unwinding from duplex siRNA to single-stranded siRNA by the dCMTR1^{W231X} mutant.

FIG. 11 illustrates results of confirming physical interaction between dCMTR1 and R2D2, which is an essential factor for initiation of holo-RISC complex formation, in which FIGS. 11A and 11C illustrate results of immunoprecipitation confirmed by western blotting, and FIG. 11B illustrates a photograph observed with a confocal microscope after immunostaining.

FIG. 12 illustrates results of confirming the binding of various deletion fragments of dCMTR1 and R2D2 by western blotting after co-immunoprecipitation to confirm a binding site between dCMTR1 and R2D2.

FIG. 13 illustrates results of confirming the binding of various deletion fragments of R2D2 and dCMTR1 by western blotting after co-immunoprecipitation to confirm a binding site between dCMTR1 and R2D2.

### BEST MODE FOR CARRY OUT THE INVENTION

The present inventors have studied a novel technology capable of enhancing the production of siRNA itself that induced gene silencing of a target gene and improving a gene silencing function, out of a conventional method of focusing only on efficient delivery and stability enhancement of siRNA in the technical field of disease treatment using siRNA, and first found that cap1 2′-O-ribose methyltransferase (CMTR1) had the activity of enhancing the production and function of siRNA.

Specifically, the present inventors induced a mutation in an X chromosome of Drosophila using EMS in vivo, identified a mutant in which white eyes are changed to yellow due to inhibition of white gene silencing in Drosophila having two GMR-wIR transgenes expressing hairpin dsRNA inducing white gene silencing as a genetic background, and identified a mutated gene through genetic mapping. As a result, it was confirmed that a CG6379 gene of Drosophila was mutated, and it was confirmed that a 2′-O-MTase domain failed to function in the mutant, which was named as dCMTR1^{W231X} in an example embodiment of the present disclosure.

Here, the GMR-wIR is a gene that enables the expression of hairpin dsRNA that induces RNAi of a white gene in Drosophila eyes, and in Drosophila having the two GMR-wIR genes, the red eyes are changed to white due to gene silencing of the white gene in the eyes.

With respect to a CG6379 gene of Drosophila identified in relation to RNAi, as a result of analyzing ortholog genes which are expected to have the same function among different species targeting a genome of eukaryote, it was confirmed that cap1 2′-O-ribose methyltransferase (CMTR1) is conserved in various eukaryotes.

Accordingly, the present inventors performed an experiment for confirming whether the enzymatic activity of cap1 2′-O-ribose methyltransferase (CMTR1) is related to RNAi caused by dsRNA and to which step is related in the RNAi pathway.

The CMTR1 methylates cap0-mRNA to 2′-O-ribose to form a cap1 structure. Accordingly, the present inventors induced overexpression of a full-length human CMTR1 (hCMTR1) gene in eye tissues with respect to a dCMTR1^{W231X} mutant with dark orange eyes in which gene silencing induced by one GMR-wIR transgene was inhibited, and as a result, found that inhibition of the white gene silencing was restored so that the dark orange eyes were changed to pale orange eyes.

On the other hand, when the overexpression of a hCMTR1^{K239A} or dCMTR1^{K179A} mutant gene expressing CMTR1 in which the 2′-O-MTase activity was lost was induced in eye tissue, the inhibition of white gene silencing was not restored.

Through these results, the present inventors found that cap1 2′-O-ribose methyltransferase (CMTR1) is involved in gene silencing, and that the cap1 methyltransferase activity by a 2′-O-methyltransferase domain of CMTR1 is involved in the RNAi mechanism.

In addition, in another example embodiment of the present disclosure, for the dCMTR1^{W231X} mutant with inhibited gene silencing, the profile change in the cap1 structure was analyzed. As a result, in the dCMTR1^{W231X} mutant, 2′-O-methyluridine 5′-monophosphate, 2′-O-methylguanosine 5′-monophosphate and 2′-O-methylcytidine 5′-monophosphate derived from the mRNA transcripts were not detected, and 2′-O-methyladenosine 5′-monophosphate was also slightly detected. On the other hand, it was confirmed that when a wild-type dCMTR1 or hCMTR1 gene was overexpressed, the level of 2′-O-ribose methylated nucleotide was increased.

Therefore, in the case of the dCMTR1^{W231X} mutant with inhibited gene silencing, that is, when the action of CMTR1 does not work properly, it indicates that the production of 2′-O-ribose methylated nucleotide is inhibited, and CMTR1 has the cap1 methyltransferase activity.

In another example embodiment of the present disclosure, as confirmed above that the CMTR1 of the present disclosure has a function of cap1 methyltransferase, in order to confirm whether the cap1 methylation of dsRNA by CMTR1 is involved in the gene silencing (RNAi), after 5′ppp (no cap)-, cap0-, or cap1-GFP dsRNA was introduced into Drosophila cells expressing GFP according to the presence of CuSO₄, gene silencing was analyzed.

As a result, a change in activity of GFP gene silencing by 5′ppp- and cap0-GFP dsRNA could not be confirmed. However, it could be confirmed that the activity of GFP gene silencing by cap1-GFP dsRNA was greatly increased.

Therefore, it could be seen that the cap1 structure of dsRNA contributes to increasing gene silencing, and it could be seen that the CMTR1 of the present disclosure forms the cap1 structure of dsRNA through the action of cap1 methyltransferase, thereby increasing gene silencing.

In addition, in order to confirm how the CMTR1 of the present disclosure specifically acts in the siRNA production and the gene silencing mechanism, the present inventors confirmed the production levels of wIR siRNA through northern blotting with respect to dCMTR1^{W231X}, a mutant with inhibited gene silencing and wild-type Drosophila (normal control).

As a result, the dCMTR1^{W231X} mutant showed a significantly reduced level of siRNA compared to the normal control, whereas when overexpressing the wild-type dCMTR1 gene in the mutant, the reduced level of siRNA was increased.

On the other hand, it was found that when the dCMTR1^{K179A} gene having a mutation losing 2′-O-MTase activity was overexpressed, the decreased level of siRNA was not increased.

Therefore, through these results, it could be seen that the CMTR1 may increase the production of siRNA for gene silencing.

In addition, in order to confirm a relationship between CMTR1 and the RNA-induced silencing complex (RISC) in the mechanism involved in siRNA-induced gene silencing, the present inventors analyzed the cleavage degree of the target mRNA by siRNA in the dCMTR1^{W231X} mutant and the wild-type normal control. As a result, it was found that the level of the cleaved target mRNA was more increased in the wild-type normal control than the mutant.

In addition, the effect of CMTR1 on the RNA-induced silencing complex (RISC) was analyzed, and as a result, it was confirmed that the dCMTR1^{W231X} mutant causes abnormalities in the formation of holo-RISC required for degradation of the target mRNA.

In general, dsRNA present in the cell is cleaved by ribonuclease called Dicer to be converted into small RNA of 21 to 23 bp, and the cleaved small RNA form is referred to as short interfering RNA (siRNA). The siRNA cleaved from the cytoplasm binds to the RNA-induced silencing complex (RISC), but RISC assembly begins with the formation of a R2D2/Dicer-2 initiator (RDI) complex by binding of duplex siRNA to an R2D2/Dicer-2 heterodimer, and then other proteins bind to the RDI complex to form a RISC-loading complex (RLC), and Argonaute-2 (Ago2) binds to the RLC to form pre-RISC. Thereafter, when the duplex siRNA is unwinding, holo-RISC capable of cleaving the target mRNA is formed.

Accordingly, the present inventors analyzed the effect of CMTR1 on the formation of holo-RISC capable of cleaving the target mRNA, and as a result, in the case of the dCMTR1^{W231X} mutant with the lost CMTR1 function, the electrical movement of the RISC complex was slowed compared to holo-RISC in native gel electrophoresis analysis.

This is because when duplex siRNA of pre-RISC is unwinding, the duplex siRNA is converted into holo-RISC capable of cleaving the target mRNA, and due to the unwinding of siRNA, the size of holo-RISC is reduced compared to that of pre-RISC to further increase the mobility in electrophoresis.

However, in the dCMTR1^{W231X} mutant, it was confirmed that since the electrical movement of the RISC complex in native gel electrophoresis was slowed compared to holo-RISC, pre-RISC was not converted to holo-RISC, and it was confirmed that the unwinding degree of duplex siRNA was reduced compared to a normal group.

This means that CMTR1 may enhance the gene silencing activity in RNAi (gene silencing) by promoting the conversion from pre-RISC to holo-RISC.

In addition, the present inventors performed co-immunoprecipitation with CMTR1 and R2D2 in Drosophila S2 cells to confirm whether CMTR1 has a physical binding action with R2D2, which is an essential factor for initiation of the formation of the holo-RISC complex.

As a result, the mutual physical binding between CMTR1 and R2D2 was confirmed, and in particular, it was found that the carboxyl-terminal region of CMTR1 and the carboxyl-terminal region of R2D2 were bound to each other.

Through these results, the present inventors found that the CMTR1 of the present disclosure interacts with R2D2, and may ultimately improve the silencing of the target gene by siRNA by promoting the conversion from pre-RISC to holo-RISC.

Therefore, the present disclosure may provide a composition for enhancing the function of siRNA including a cap1 2′-O-ribose methyltransferase (CMTR1) protein as an active ingredient.

In the present disclosure, the CMTR1 may consist of an amino acid sequence of SEQ ID NO: 1 or SEQ ID NO: 5, in which SEQ ID NO: 1 is an amino acid sequence for a Drosophila CMTR1 protein, and SEQ ID NO: 5 is an amino acid sequence for a human CMTR1 protein.

In addition, the CMTR1 protein of SEQ ID NO: 1 is encoded by a nucleotide sequence of SEQ ID NO: 2, and the CMTR1 protein of SEQ ID NO: 5 may be encoded by a nucleotide sequence of SEQ ID NO: 6, but are not limited thereto, and the homologues of the sequences may be included within the scope of the present disclosure.

Specifically, the CMTR1 may include at least 70%, more preferably at least 80%, even more preferably at least 90%, most preferably at least 95% sequence homology with the nucleotide sequence of SEQ ID NO: 2 or SEQ ID NO: 6, respectively.

The “% of sequence homology” to the polynucleotide is determined by comparing two optimally arranged sequences with a comparison region, and a part of a polynucleotide sequence in the comparison region may include addition or deletion (i.e., gap) compared with a reference sequence (not including addition or deletion) for the optimal alignment of the two sequences.

The CMTR1 of the present disclosure may increase the production of siRNA through cap1 methylation of dsRNA on an RNAi pathway by dsRNA, and promote the formation of a holo-RNA-induced silencing complex (RISC) capable of cleaving the target mRNA for gene silencing.

The carboxyl-terminal region of the CMTR1 may mutually bind to a carboxyl-terminal region of R2D2 included in the holo-RISC complex to promote the conversion of pre-RISC to holo-RISC.

Here, the carboxyl terminal region of CMTR1 mutually binding to the R2D2 is a region consisting of the 388th to 788th amino acid sequence of SEQ ID NO: 1, which corresponds to a region consisting of the 1162nd to 2367th nucleotide sequence of SEQ ID NO: 2.

Here, the carboxyl terminal region of R2D2 mutually binding to the CMTR1 is a region consisting of the 237th to 311th amino acid sequence of SEQ ID NO: 3, which corresponds to a region consisting of the 709th to 936th nucleotide sequence of SEQ ID NO: 4.

The CMTR1 contained in the composition may also be included in the form of a protein, or may also be included in the form of a gene encoding the CMTR1 amino acid sequence of SEQ ID NO: 1 or SEQ ID NO: 5 which is inserted into an expression vector.

Further, the present disclosure may provide a composition for enhancing the gene silencing activity by siRNA including a cap1 2′-O-ribose methyltransferase (CMTR1) protein as an active ingredient.

As described above, it was confirmed that the CMTR1 of the present disclosure may induce cap1 methylation and have the activity to increase the production of siRNA for gene silencing, and it was confirmed that the CMTR1 of the present disclosure may increase the silencing activity of the target gene through cleavage of the target mRNA by promoting the conversion of pre-RISC to holo-RISC.

Therefore, the composition including the CMTR1 of the present disclosure may enhance the target gene silencing activity by siRNA.

Further, the present disclosure may provide a method for producing siRNA for gene silencing in vitro including treating a cell with an expression vector containing a gene encoding a CMTR1 amino acid sequence of SEQ ID NO: 1 or SEQ ID NO: 5.

That is, by introducing the expression vector inserted with the CMTR1 gene into a cell in vitro, it is possible to mass-produce siRNA for gene silencing through the overexpression induction of CMTR1 in the cell.

The composition for enhancing the production of siRNA and the composition for enhancing the gene silencing activity by siRNA according to the present disclosure may be particularly useful in the development of a therapeutic agent using siRNA.

### MODE(S) FOR CARRYING OUT THE INVENTION

Hereinafter, the present disclosure will be described in more detail with reference to Example embodiments. However, these Example embodiments are more specifically illustrative the present disclosure, and the scope of the present disclosure is not limited to these Example embodiments.

### Preparation Examples and Experimental Methods

Materials

All flies were raised using standard cornmeal/agar media at 25° C. In addition, a dCMTR1^{W231X} mutant was isolated from a genetic screen method based on mosaic analysis of an X chromosome with FRT19A for an adult eye in a GMR-wIR genetic background. A FRT19A strain was used as a wild-type, and a w¹¹¹⁸ strain was also used as a control. dcr-2^{L811fsX}, Ago2⁴¹⁴, and Ago2^{V966M} alleles have been described in Kim et al., 2007; Lee et al., 2004; Okamura et al., 2004, and the Ago2⁴¹⁴ strain was provided by M. Siomi (University of Tokyo, Tokyo, Japan).

Preparation of Transgenic Drosophila

To generate transgenic Drosophila expressing wild-type dCMTR1 or hCMTR1 via a GAL4/UAS system, full-length cDNAs for each gene were synthesized by reverse transcription and PCR methods using primers of Table 1 below, and then inserted into a pUAST vector using NotI and XbaI restriction enzyme sites.

TABLE 1

| Name | Sequence (5′ to 3′) | [symbol] |
|---|---|---|
| [symbol] | AGCTTTCTTGGGCACTCCCAG | [symbol] |
| [symbol] | CACAAGTTCGGATTTACGGGTT | [symbol] |
| [symbol] | TGAGGTAGTAGGTTGTATAGT | [symbol] |
| [symbol] | GCAAGAACTCAGACTGTGATG | [symbol] |
| [symbol] | TACAACCCTCAACCATATGTAGTCCAAGCA | [symbol] |
| [symbol] | GAGATGCAGGCCAGGTGCGC | [symbol] |
| [symbol] | GGCGGTAAAGGAGTCCAGTC | [symbol] |
| [symbol] | p-CGUACGCGGAAUACUUCGAUU-OH | [symbol] |
| [symbol] | p-UGUACGCGGAAUACUUCGAUU-OH | [symbol] |
| [symbol] | p-UCGAAGUAUUCCGCGUACGUG-OH | [symbol] |
| [symbol] | AAGCGGCCGCATGGACCAACCTTCGGACGATG | Cloning |
| [symbol] | AATCTAGACTAGCTGTGGCCCAACTTGTCC | Cloning |
| [symbol] | AAGCGGCCGCATGAAGAGGAGAACTGACCC | Cloning |
| [symbol] | AATCTAGATTAGGCCCTGTGCATCTGGA | Cloning |
| [symbol] | AATCTAGAAGACGAACCTTCGGACGATGA | Cloning |
| [symbol] | AAAAGCTTCTAGCTGTGGCCCAACTTGT | Cloning |
| [symbol] | AAGAATTCGTGAGCAAGGGCGAGGAGCTGT | Cloning |
| [symbol] | AAGCGGCCGCCTAGAAGCTTGAGCTCGAGA | Cloning |
| [symbol] | AAACTAGTATGTACCCATACGATGTTCCTGAC | Cloning |
| [symbol] | AAGAATTCTTAGCAGCGTAATCTGGAACGTCATA | Cloning |
| [symbol] | AAGAATTCCCTGCCTGCGAGGAGCTC | Cloning |
| [symbol] | AAGAATTCGCGGAGACGGCGGGCATC | Cloning |
| [symbol] | AAGCGGCCGCCTAGCTGTGGCCCAAG | Cloning |
| [symbol] | AAGAATTCGACGAACCTTCGGACGAT | Cloning |
| [symbol] | AAGCGGCCGCCTACGCATCCGAACGT | Cloning |
| [symbol] | TAATACGACTCACTATAGGGCATTATCCGAACCATCG | [symbol] |
| [symbol] | TAATACGACTCACTATAGGGCAGAACTGGCGATCGTTCG | [symbol] |
| [symbol] | TAATACGACTCACTATAGGGAGACAAGGGCGAGGAGCTGTT | [symbol] |
| [symbol] | TGCTCAGGTAGTGGTTGTCG | [symbol] |
| [symbol] | TAATACGACTCACTATAGGGAGACTTGTACAGCTCGTCCATGC | [symbol] |
| [symbol] | CCTGAAGTTCATCTGCACCA | [symbol] |
| [symbol] | GATGTTGGCCATCGCGACGGCGGCGCGG | [symbol] |
| [symbol] | CCGCGCCGCCGTCGCGATGGCCAACATC | [symbol] |
| [symbol] | TACAAAATCCATGTTAGCCATCGCCATTGCTGCCCTGTTTAGAAAG | [symbol] |
| [symbol] | CTTTCTAAACAGGGCAGCAATGGCGATGGCTAACATGGATTTTGTA | [symbol] |
| [symbol] | p- [symbol] CGAAGUAUUCCGCGUACCUG-OH | [symbol] |
| [symbol] | p- [symbol] CGAAGUAUUCCGCGUACCUG-OH | [symbol] |
| [symbol] | p- [symbol] GUACGCGGAAUACUUCGUUU-OH | [symbol] |
| [symbol] | p- [symbol] GUACGCGGAAUACUUCGUUU-OH | [symbol] |

[symbol]

[symbol]

indicates data missing or illegible when filed

In addition, site-directed mutagenesis was performed using PfuUltra High-Fidelity DNA polymerase (Stratagene, San Diego, Calif., USA) and full-length cDNA for dCMTR1 or hCMTR1 to prepare UAS-transgene lines expressing cap1 MTase-dead variants (dCMTR1^{K179A} or hCMTR1^{K239A}). Primer sets used for site-directed mutagenesis are disclosed in Table 1 above. Each prepared DNA construct was co-microinjected into w¹¹¹⁸ embryos with P{Δ2-3} expressing transposase. The expression of wild-type R2D2 or its deletion derivatives with an amino-terminal triple-FLAG epitope was induced in S2 cells. In addition, a green fluorescent protein (GFP) with either an amino-terminal triple-HA or FLAG epitope expressed in S2 cells was used as a heterologous control, and a GFP coding sequence was amplified by a PCR method using the primer sets of Table 1 above, and cloned with a pMT-HA or pMT-FLAG vector using SpeI/EcoRI restriction enzyme sites. Wild-type dCMTR1 or its deletion derivatives with an amino-terminal triple-HA epitope was expressed in S2 cells, full-length cDNA of dCMTR1 or its deletion variants were amplified by PCR using the primers of Table 1 above, and cloned with a pMT-HA vector using the EcoRI/NotI restriction enzyme sites.

Eye Pigment Assay

An eye pigment was obtained from 30 adult Drosophila, homogenized with 0.01 M HCl-containing ethanol, and then incubated at 65° C. for 10 minutes. After centrifugation, a supernatant of each sample was obtained and then the absorbance at 480 nm was measured.

In Vitro RNAi and siRNA Unwinding Assays

The preparation of an embryo extract and cleavage of a target RNA were performed by methods known by Pham and the like. RISC assembly was performed by native gel electrophoresis using a Pp-luc siRNA duplex containing a G:U wobble at a 5′ end of a sense strand and highly asymmetric for retention of a guide strand in Ago2. The siRNA unwinding assay was performed using the asymmetric Pp-luc siRNA duplex, and the sequences of the sense and antisense strands of Pp-luc siRNAs were disclosed in Table 1 above.

RNA Analysis

Hairpin RNA expressed from GMR-wIR was detected by northern blotting, and at this time, a 5′-end labeled DNA probe synthesized by PCR using the hp-w(Ex3)-F and hp-w(Ex3)-R primers of Table 1 above was used. Small RNAs were detected by northern blotting Northern blotting using 5′-end labeled DNA oligonucleotides.

Western Blotting

Western blot was performed using antibodies of anti-dCMTR1 (1:200 dilution; Abmart, Berkeley Heights, N.J., USA), anti-hCMTR1 (1:1000 dilution; Abcam, Cambridge, UK), anti-R2D2 (1:1000 dilution; a gift from M. Siomi), anti-FLAG (1:1000 dilution; Sigma, St. Louis, Mo., USA), anti-HA (1:1000 dilution; Sigma), anti-Dcr-2 (1:1000 dilution; Abcam), anti-Dcr-1 (1:500 dilution; Abcam), anti-Ago2 (1:3 dilution; a gift from M. Siomi), anti-Ago1 (1:1000 dilution; Abcam), anti-dFMR (1:1000 dilution; a gift from G. Hannon, University of Cambridge, Cambridge, UK) and anti-VIG (1:1000 dilution; a gift from G. Hannon). In addition, anti-β-actin (1:1000 dilution; Santa Cruz Biotechnology, Dallas, Tex., USA) was used as a loading control.

Thin-Layer Chromatography (TLC) Analysis

Total RNA was extracted from 20 adult Drosophila per genotype or from 100 adult Drosophila per genotype using a TRIzol reagent. Then, a magnetic mRNA isolation kit (New England Biolabs, Ipswich, Mass., USA) was used to purify mRNA from total RNA. The purified mRNA was decapped with RNA 5′ pyrophosphohydrolase (New England Biolabs) at 37° C. for 1 hour and then treated with FastAP thermosensitive alkaline phosphatase and additionally reacted at 37° C. for 10 minutes. Thereafter, phenol-chloroform extraction and ethanol precipitation were performed, and then the 5′ ends of the RNA were radiolabeled using T4 polynucleotide kinase (PNK; New England Biolabs) and [γ-³²P]-ATP by reacting at 37° C. for 30 minutes. In addition, synthetic RNA oligonucleotides of Table 1 above were also radiolabeled using T4 PNK and [γ-³²P]-ATP. Thereafter, PNK was inactivated by heat treatment, and the unlabeled radioisotopes were removed using RNA Clean & Concentrator-5 (Zymo Research, Irvine, Calif., USA). Thereafter, the ³²P-labeled RNA was completely degraded into 5′-monophosphate nucleosides by reacting at 37° C. for 2 hours using Nuclease P1 (Sigma). The mononucleotides were developed 2-dimensionally, in which solvent A was used in one direction and then either solvent B or C was used in a direction perpendicular to the first direction. At this time, the compositions of the used solvents were as follows.

Solvent A: isobutyric acid/25% ammonia/water [66:1:33 (v:v:v)]

Solvent B: 0.1 M sodium phosphate buffer (pH 6.8)/ammonium sulfate/1-propanol [100:60:2 v:v:v]

Solvent C: isopropanol/concentrated HCl/water [68:18:14 (v:v:v)]

Thereafter, the TLC plate was air-dried, and spots were visualized by autoradiography.

Cell Culture

Drosophila S2 cells and a S2 cell line with a copper sulfate-inducible GFP expression were incubated at 25° C. in Schneider's Drosophila media (GIBCO, Gaithersburg, Md., USA) containing 10% fetal bovine serum (HyClone, Logan, Utah, USA) and 100 U/mL penicillin-streptomycin (HyClone).

RNAi-Mediated Gene Knockdown

5′ppp-, cap0-, or cap1-GFP dsRNAs, and DNA templates for GFP sense (602 nucleotides) and antisense (586 nucleotides) strands of dsRNA were amplified and prepared by PCR using primer sets of Table 1 above. The PCR products were used for in vitro transcription reaction in the presence of [α-³²P]-UTP using T7 RNA polymerase (New England Biolabs), and the transcripts were gel-purified and used to produce 5′ppp-GFP dsRNAs. Aliquots of each RNA strand were capped with 7-methylguanosine on the first transcribed nucleotide using the Vaccinia Capping System (New England Biolabs), and then used to produce cap0-GFP dsRNAs. Cap0-methylated RNA strands were purified using RNA Clean & Concentrator-5. Next, 2′-O-ribose methylation was added to the first transcribed nucleotide modified by cap0 methylation using a ScriptCap 2′-O-Methyltransferase kit (CELLSCRIPT, Madison, Wis., USA) and the produced RNAs were used to produce cap1-GFP dsRNAs. Complementary strands were mixed in a 1:1 ratio in an annealing buffer [30 mM HEPES-KOH (pH 7.5), 100 mM potassium acetate, and 2 mM magnesium acetate], heated at 95° C. for 1 minute, and then slowly cooled to room temperature to produce 5′ppp-, cap0-, or cap1-GFP dsRNAs. DsRNA against LacZ was also prepared using gene-specific primers of Table 1 above.

GFP expression in S2 cells was induced by the addition of CuSO₄ of 0.7 mM, and after 24 hours of expression induction, the cells (1×10⁶ cells/well) were transfected with 2 μg of mock- or each dsRNA using Lipofectamine 2000 (Invitrogen). After 12 hours, the cells were dissolved in a cold lysis buffer [20 mM Tris-HCl (pH 8.0), 150 mM NaCl, 5 mM MgCl₂, 2 mM DTT, 0.1% Triton X-100 and protease inhibitor cocktail (Roche, Basel, Switzerland)] and then western blotting was performed.

Co-Immunoprecipitation (Co-IP) and RNase Treatment

Co-immunoprecipitation (Co-IP) was performed by the following method. S2 cells were co-transfected with DNA expressing two proteins tagged with different epitope tags using a FuGENE HD Transfection reagent (Promega, Madison, Wis., USA). The gene expression was induced by the addition of CuSO₄ of 0.7 mM, and after transfection for 48 hours, cell lysates were prepared using the used cold cell lysis buffer. Thereafter, supernatants (500 μg) were isolated by centrifugation at a rate of 15,000×g and then used for immunoprecipitation.

The RNase treatment was performed by the following method, but the supernatants of the obtained cell lysates were added with 20 μL RNase A/T1 mix (Thermo Fisher Scientific) prior to the addition of antibodies and reacted at 37° C. for 15 minutes. The respective samples subjected to the immunoprecipitation were analyzed by western blotting.

Immunofluorescence Cell Staining

S2 cells were co-transfected with DNA constructs for expressing HA-tagged dCMTR1 and FLAG-tagged R2D2 using a FuGENE HD Transfection reagent. Thereafter, expression was induced by the addition of CuSO₄ of 0.7 mM, and after transfection for 48 hours, the cells were reacted with anti-FLAG (1:200 dilution; Sigma) and anti-HA (1:200 dilution; Sigma) antibodies as primary antibodies and then reacted with secondary Alexa-Fluor-488- and Alexa-Fluor-594-conjugated antibodies (1:500 dilution for each; Molecular Probes, Mulgrave, VIC, Australia), respectively. Thereafter, samples were placed in a Vectashield mounting medium containing 4′,6-diamidino-2-phenylindole (DAPI; Vector Laboratories, Stonesfield, UK) and then observed using a confocal laser scanning microscope (Carl Zeiss, Oberkochen, Germany)

### Example Embodiment 1

Identification and Confirmation of CMTR1 as an RNAi Positive Regulator

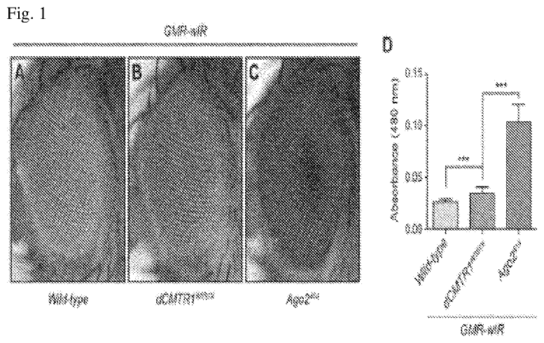

To identify novel genes related with gene silencing through siRNA, an EMS-induced mutant was screened on a Drosophila X chromosome having a GMR-wIR transgene as a genetic background, which expressed hairpin dsRNA to induce silencing of a white gene in Drosophila eyes. As a result of EMS-induced mutation screening analysis, a homozygous mutant of an RNAi positive regulator of which the eye color was changed from a white color to a yellow color by partial suppression of the white gene silencing by two copies of GMR-wIR was identified (see FIG. 1).

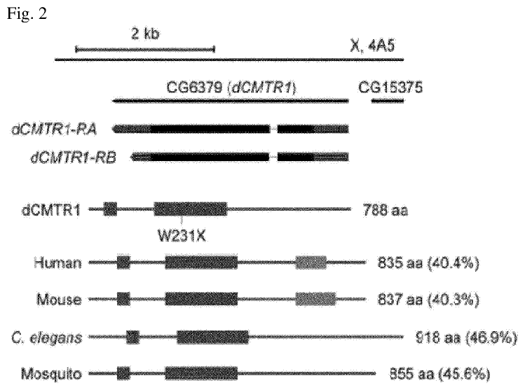

In addition, the present inventors confirmed a mutated gene in a mutant identified through genetic mapping, which was a CG6379 gene, and furthermore confirmed that the CG6379 gene was an ortholog of cap1 2′-O-ribose methyltransferase (CMTR1; cap1 MTase) which was evolutionarily conserved in various eukaryotes including humans. In addition, the CG6379 gene identified in the present disclosure had a 2′-O-MTase domain, and as a DNA sequencing analysis result, it was confirmed that the identified mutant had a premature stop codon W231X in a 2′-O-MTase domain (see FIG. 2).

In addition, since the mutant did not function as the 2′-O-MTase domain, and had the same phenotype as a dCMTR1 null mutant, the present inventors named the identified mutant as a dCMTR1^{W231X} mutant.

### Example Embodiment 2

Association Analysis with Cap1 2′-O-MTase Activity on dsRNA-Triggered RNAi Pathway

In higher eukaryotes, CMTR1 allows mRNA to form a cap1 structure by 2′-O-ribose methylation of cap0-mRNAs. Thus, the present inventors specifically induced overexpression of a CMTR1 protein in an eye tissue of the dCMTR1^{W231X} mutant using a Drosophila GAL4/UAS system, in order to confirm whether the loss of the cap1 MTase function in the dCMTR1^{W231X} mutant affected GMR-wIR-induced white gene silencing.

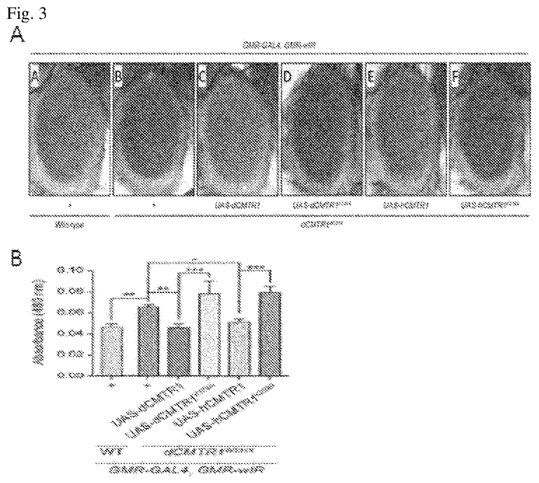

As a result, it was confirmed that overexpression of a human CMTR1 (hCMTR1) protein by GMR-GAL4 restored (recovered) the inhibition of white gene silencing caused by mutation of the dCMTR1 gene in the eye tissue of the dCMTR1^{W231X} mutant (see FIG. 3). Meanwhile, overexpression of CMTR1 catalytic dead proteins (hCMTR1^{K239A} and dCMTR1^{K179A}) by GMR-GAL4 failed to restore the inhibition of white gene silencing caused by mutation of the dCMTR1 gene in the eye tissue of the dCMTR1^{W231X} mutant (see FIG. 3). These results indicate that cap1 MTase activity is involved in the RNAi pathway triggered by dsRNA in Drosophila.

In addition, the present inventors performed two-dimensional thin-layer chromatography (2D-TLC) analysis to determine whether the profile of the cap1 structure of the dCMTR1^{W231X} mutant was altered due to the loss of the cap1 MTase function of the dCMTR1^{W231X} mutant.

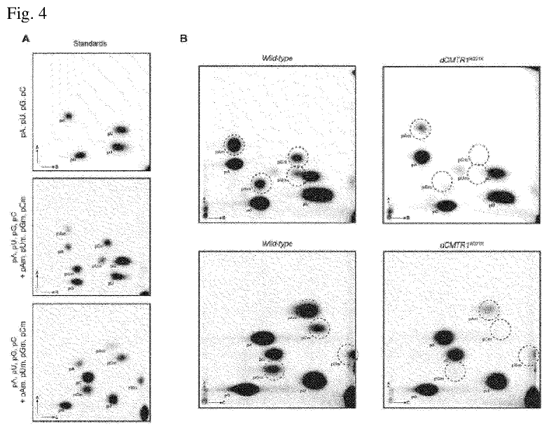

As a result, there was no detectable level of 2′-O-methyluridine 5′-monophosphate, 2′-O-methylguanosine 5′-monophosphate and 2′-O-methylcytidine 5′-monophosphate derived from the mRNA transcripts of the dCMTR1^{W231X} mutant, and 2′-O-methyladenosine 5′-monophosphate showed a relatively sharp decrease compared to the 2D-TLC result of the wild-type control. Therefore, it could be seen from these results that 2′-O-ribose methylated nucleotides were not properly produced in the dCMTR1^{W231X} mutant (see FIG. 4).

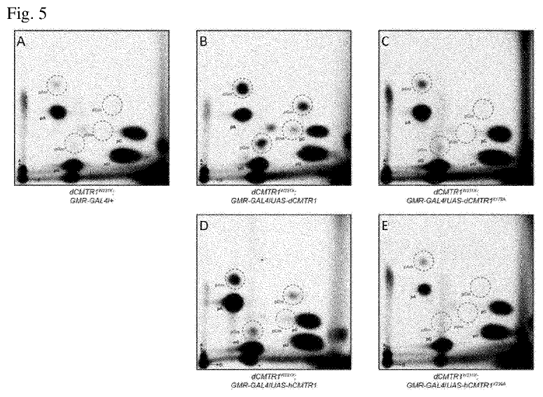

In addition, as a result of specifically inducing the overexpression of wild-type dCMTR1 and human CMTR1 cDNA in the eye tissue of the dCMTR1^{W231X} mutant using the Drosophila GAL4/UAS system, it was confirmed that the level of 2′-O-ribose methylated nucleotides was increased (see FIG. 5). These results indicate that the dCMTR1 gene has cap1 MTase activity similar to that of mammalian orthologs.

### Example Embodiment 3

Confirmation of Increase in Gene Silencing According to Cap1 Structure of dsRNA

To verify whether the cap1 methylation of dsRNA by dCMTR1 was involved in RNAi, GFP-expressing Drosophila S2 cells were transfected with 5′ppp-, cap0-, or cap1-GFP dsRNAs according to the presence or absence of CuSO₄. The Drosophila S2 cells expressing GFP were transfected with plasmid DNA in which a GFP cDNA sequence was inserted into a pMT vector having a CuSO₄-inducible promoter, and then made into a stabilized cell line to express the GFP according to the presence or absence of CuSO₄.

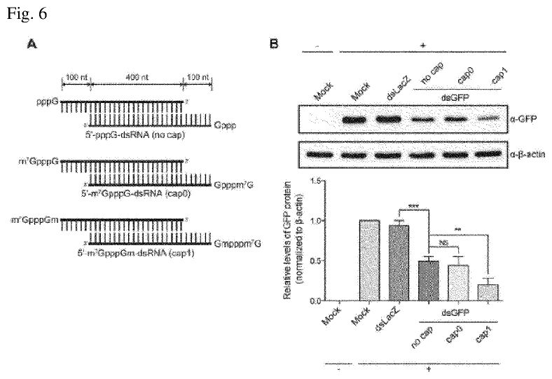

As a result of the analysis, there was no difference in silencing activity of the GFP gene of the 5′ppp- and cap0-GFP dsRNAs. However, the silencing activity of the GFP gene of cap1-GFP dsRNA was significantly increased (see FIG. 6).

Accordingly, through these result, it could be seen that the cap1 structure of dsRNA had the activity of enhancing the gene silencing of the gene in the Drosophila S2 cells.

### Example Embodiment 4

Confirmation of siRNA Abundance Regulation of dCMTR1

To examine whether cap1 methylation by dCMTR1 was involved in siRNA production, the level of siRNA produced from GMR-wIR hairpin dsRNA was measured using northern blotting.

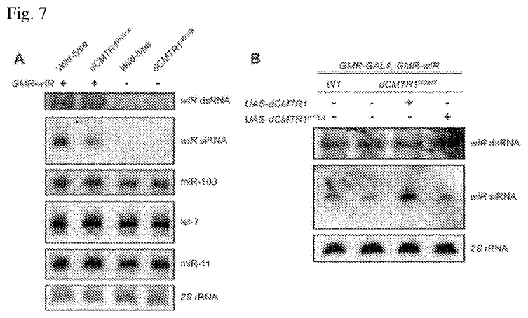

As a result, the dCMTR1^{W231X} mutant exhibited a reduction in the wIR siRNA level compared to the wild-type control (see FIG. 7A). Meanwhile, it was shown that the reduction in the wIR siRNA level in the dCMTR1^{W231X} mutant was restored again by overexpressing wild-type dCMTR1 using the GAL4/UAS system of Drosophila, and it was shown that when dCMTR1^{K179A}, a catalytic dead protein of dCMTR1, was overexpressed, the reduction in the wIR siRNA level was not restored (see FIG. 7B). Therefore, these results indicated that the catalytic activity of dCMTR1 was involved in the biosynthesis of siRNA.

### Example Embodiment 5

Analysis of Effect of dCMTR1 on Downstream after siRNA Production

The present inventors performed an experiment to confirm whether dCMTR1 also affected the downstream of RNAi after the production of siRNA. Specifically, in vitro cleavage of siRNA-triggered target mRNA was confirmed to see whether dCMTR1 was associated with the downstream after siRNA production, such as RISC assembly and RISC-directed cleavage of the target mRNA.

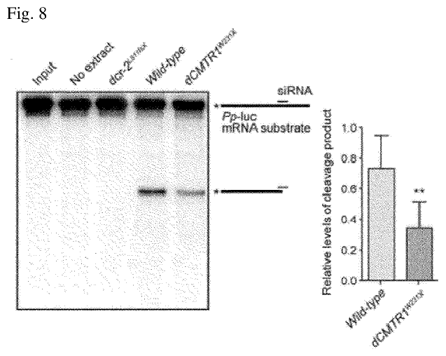

As a result, it was shown that the target cleavage activity was reduced in the embryo lysate of the dCMTR1^{W231X} mutant compared to the lysate of the wild-type control (see FIG. 8).

In addition, in order to confirm whether the reduction in target cleavage activity by the dCMTR1^{W231X} mutant had a problem in the formation of holo-RISC required for the cleavage of the target mRNA, the RNAi complex was analyzed for the RISC assembly through native gel electrophoresis.

The RISC assembly in Drosophila begins with the binding of duplex siRNA to a R2D2/Dicer-2 heterodimer to form an R2D2/Dicer-2 initiator (RDI) complex, and then, various other proteins bind to the RDI complex to form a RISC-loading complex (RLC) and Ago2 binds to RLC to form pre-RISC. When the duplex siRNA of the formed pre-RISC is unwound, a holo-RISC capable of cleaving the target mRNA is formed.

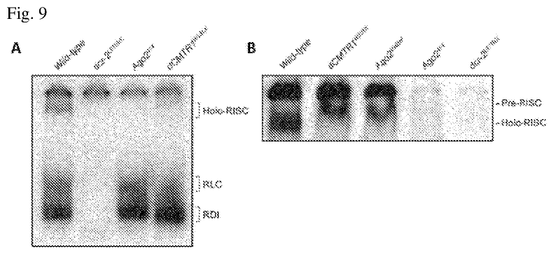

Considering this aspect, in the case of the dCMTR1^{W231X} mutant, it was confirmed that the mobility of holo-RISC was slowed in the gel electrophoresis relative to the wild-type control (see FIG. 9A). During the RISC assembly, an Ago2^{V966M} mutant lacking the Ago2 slicer activity with a problem in the unwinding activity of duplex siRNA formed pre-RISC containing duplex siRNA rather than holo-RISC containing single-stranded guide siRNA. Since pre-RISC does not be converted to holo-RISC by releasing the siRNA passenger strand, the pre-RISC has slower electrical mobility on native gel electrophoresis than holo-RISC.

In addition, as a result of performing the native gel electrophoresis on the RNAi complex including the lysates of the Ago2^{V966M} mutant, it was confirmed that the dCMTR1^{W231X} mutant expression group had similar mobility to the Ago2^{V966M} mutant expression group (see FIG. 9B).

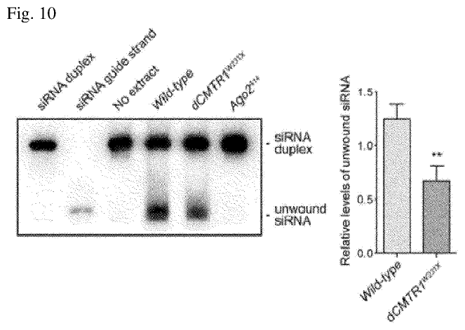

Therefore, it was found that the dCMTR1^{W231X} mutant expression group had a problem in conversion from pre-RISC to holo-RISC during RISC formation, similarly to the Ago2^{V966M} mutant expression group. In addition, it was confirmed that the dCMTR1^{W231X} mutant expression group significantly reduced the degree of unwinding of duplex siRNA compared to the wild-type control (see FIG. 10).

Therefore, through these results, the present inventors found that dCMTR1 played an important role in the RNAi downstream after siRNA production by promoting the conversion to holo-RISC from pre-RISC.

### Example Embodiment 6

Confirmation of Physical Interaction Between dCMTR1 and R2D2

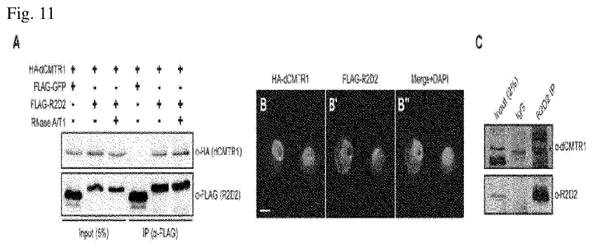

To confirm the interaction of dCMTR1 with various RNAi-related proteins, HA-tagged dCMTR1 and FLAG-tagged RNAi-related proteins were expressed in Drosophila S2 cells, and then co-immunoprecipitation (co-IP) was performed. As a result of precipitation of FLAG-tagged RNAi-related proteins using an anti-FLAG antibody, only FLAG-R2D2 showed that HA-dCMTR1 was co-immunoprecipitated. In addition, it was shown that the interaction between HA-dCMTR1 and FLAG-R2D2 was not affected even when RNase A/T1 was treated before immunoprecipitation (see FIG. 11A).

Through this, it could be seen that the interaction between HA-dCMTR1 and FLAG-R2D2 was independent of the presence or absence of RNA.

In addition, it was confirmed that HA-dCMTR1 was colocalized with FLAG-R2D2 in both the nucleus and cytoplasm of Drosophila S2 cells (see FIG. 11B). To confirm the interaction between R2D2 and dCMTR1, co-immunoprecipitation was also performed using anti-R2D2 and anti-dCMTR1 antibodies.

As a result, it was confirmed that endogenous dCMTR1 physically interacts with endogenous R2D2 in Drosophila S2 cells. Through these results, it was found that dCMTR1 interacted with R2D2, a protein constituting the RDI complex that initiated RISC assembly (see FIG. 11C).

In addition, in order to confirm the interaction positions of dCMTR1 and R2D2, where the interaction was confirmed, in Drosophila S2 cells, each deletion protein fragment conjugated with an affinity tag was expressed, and then co-immunoprecipitated. The dCMTR1 protein largely consists of a Glycine rich domain, G-patch domain, an RNA methyltransferase domain, and a carboxyl-terminal region. The dCMTR1 deletion protein fragment was constructed by deleting each domain, and HA was used as an affinity tag. In addition, the R2D2 protein largely consists of two double-stranded RNA-binding domains (dsRBDs) and a carboxyl-terminal region. The R2D2 deletion protein fragment was constructed by deleting each domain, and FLAG was used as an affinity tag. HA-tagged dCMTR1 and dCMTR1 deletion protein fragments were expressed in Drosophila S2 cells expressing the FLAG-R2D2 protein, respectively, and then co-immunoprecipitation was performed.

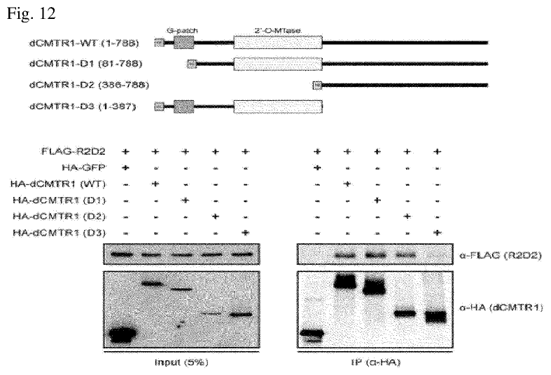

As a result, it was found that when the G-patch and RNA methyltransferase domains were deleted in the dCMTR1 protein, the interaction with the FLAG-R2D2 protein was not affected. Meanwhile, when the carboxyl-terminal region was deleted, the interaction with the FLAG-R2D2 protein was significantly reduced (see FIG. 12).

These results indicated that the carboxyl-terminal region of dCMTR1 was very important for interaction with R2D2.

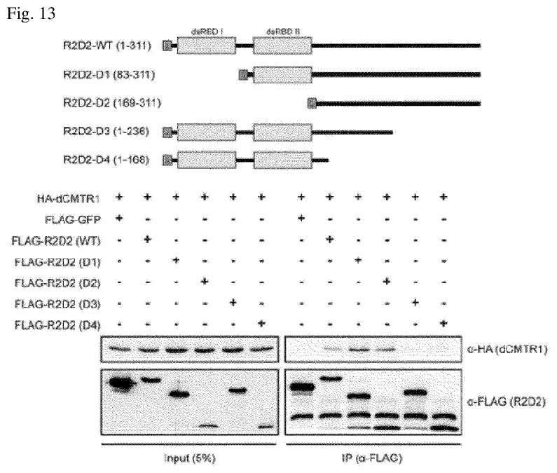

Furthermore, even in the case of the R2D2 protein, it was found that the interaction with the HA-dCMTR1 protein was reduced only in the case of two types of FLAG-R2D2 deletion protein fragments in which the carboxyl-terminal region was deleted. Furthermore, it was confirmed that carboxyl-terminal residues 237 to 311 of R2D2 were critical for interaction with dCMTR1 (see FIG. 13). Through this, it was confirmed that dCMTR1 and R2D2 interacted with each other through their carboxyl-terminal regions.

The present disclosure has been described above with reference to preferred embodiments thereof. It will be understood to those skilled in the art that the present disclosure may be implemented as a modified form without departing from an essential characteristic of the present disclosure. Therefore, the disclosed example embodiments should be considered in an illustrative viewpoint rather than a restrictive viewpoint. The scope of the present disclosure is illustrated by the appended claims rather than by the foregoing description, and all differences within the scope of equivalents thereof should be construed as being included in the present disclosure.

1. A composition for enhancing the production of siRNA comprising a cap1 2′-O-ribose methyltransferase (CMTR1) protein as an active ingredient.

2. The composition for enhancing the production of siRNA of claim 1, wherein the CMTR1 protein consists of an amino acid sequence of SEQ ID NO: 1 or SEQ ID NO: 5.

3. The composition for enhancing the production of siRNA of claim 1, wherein the CMTR1 protein is encoded by a nucleotide sequence of SEQ ID NO: 2 or SEQ ID NO: 6.

4. The composition for enhancing the production of siRNA of claim 1, wherein on an RNAi pathway triggered by dsRNA, the CMTR1 increases the production of siRNA from dsRNA through formation of a cap1 structure by 2′-O-ribose methylation and increases the gene silencing activity by promoting the formation of a holo-RNA-induced silencing complex (RISC) capable of cleaving a target mRNA for gene silencing.

5. The composition for enhancing the production of siRNA of claim 1, wherein a carboxyl-terminal region of the CMTR1 binds to a carboxyl-terminal region of R2D2 in the holo-RISC complex.

6. The composition for enhancing the production of siRNA of claim 5, wherein the carboxyl-terminal region of the CMTR1 consists of the 388th to 788th amino acid sequence of SEQ ID NO: 1, and the carboxyl-terminal region of the R2D2 consists of the 237th to 311th amino acid sequence in a R2D2 amino acid sequence of SEQ ID NO: 3.

7. The composition for enhancing the production of siRNA of claim 1, wherein in the cap1 2′-O-ribose methyltransferase (CMTR1), a gene encoding the CMTR1 amino acid sequence of SEQ ID NO: 1 or SEQ ID NO: 5 is inserted into an expression vector.

8. A composition for enhancing the gene silencing activity by siRNA comprising a cap1 2′-O-ribose methyltransferase (CMTR1) protein as an active ingredient.

9. The composition for enhancing the gene silencing activity by siRNA of claim 8, wherein the CMTR1 protein consists of an amino acid sequence of SEQ ID NO: 1 or SEQ ID NO: 5.

10. The composition for enhancing the gene silencing activity by siRNA of claim 8, wherein the CMTR1 protein is encoded by a nucleotide sequence of SEQ ID NO: 2 or SEQ ID NO: 6.

11. The composition for enhancing the gene silencing activity by siRNA of claim 8, wherein on an RNAi pathway triggered by dsRNA,
the CMTR1 increases the production of siRNA from dsRNA through formation of a cap1 structure by 2′-O-ribose methylation to enhance the gene silencing activity, and
a carboxyl-terminal region of the CMTR1 binds to a carboxyl-terminal region of the R2D2 in the holo-RISC complex to promote the formation of a holo-RNA-induced silencing complex (RISC) capable of cleaving a target mRNA for gene silencing.

12. The composition for enhancing the gene silencing activity by siRNA of claim 11, wherein the carboxyl-terminal region of the CMTR1 consists of the 388th to 788th amino acid sequence of SEQ ID NO: 1, and the carboxyl-terminal region of the R2D2 consists of the 237th to 311th amino acid sequence in R2D2 amino acid sequence of SEQ ID NO: 3.

13. A method for producing siRNA for gene silencing in vitro comprising treating a cell with an expression vector containing a gene encoding a CMTR1 amino acid sequence of SEQ ID NO: 1 or SEQ ID NO: 5.

14. The method for producing siRNA for gene silencing in vitro of claim 13, wherein the gene consists of a nucleotide sequence of SEQ ID NO: 2 or SEQ ID NO: 6.
